(19) United States
(12) Patent Application Publication
(10) Pub. No.: US 2025/0235316 A1
(43) Pub. Date: Jul. 24, 2025

(54) MITRAL OR TRICUSPID REPAIR SYSTEMS WITH MULTI-DIRECTIONAL ANCHORS

(71) Applicant: Valcare Medical, Inc., Wilmington, DE (US)

(72) Inventors: Nadav YELLIN, Ramat Gan (IL); Guy ROGEL, Haifa (IL); Yoav ROZEN, Benyamina (IL); Raphael BENARY, Tel Aviv (IL); Shuki PORATH, Haifa (IL); Samuel M. SHAOLIAN, Newport Beach, CA (US)

(73) Assignee: Valcare Medical, Inc., Wilmington, DE (US)

(21) Appl. No.: 19/174,702

(22) Filed: Apr. 9, 2025

Related U.S. Application Data
(63) Continuation of application No. 18/156,795, filed on Jan. 19, 2023, now Pat. No. 12,279,955.
Application No. 18/156,795 is a continuation of application No. 15/923,677, filed on Mar. 16, 2018, now Pat. No. 11,576,779.
(60) Provisional application No. 62/472,633, filed on Mar. 17, 2017.

(51) Int. Cl. A61F 2/24; A61B 17/00; A61B 17/04; A61B 17/064

(52) U.S. Cl. CPC A61F 2/2448; A61B 17/064; A61F 2/2445; A61F 2/2466; A61B 2017/00783; A61B 17/0401; A61B 2017/0641; A61F 2/2427; A61F 2220/0016; A61F 2220/0025

(57) ABSTRACT

Prosthetic ring valve assemblies are disclosed. A prosthetic valve ring assembly includes an outer tube and a plurality of anchors. The outer tube includes a plurality of windows. The plurality of anchors are positioned inside the outer tube and about a perimeter of the outer tube. The plurality of anchors are configured to be emitted from the plurality of windows in order to anchor the prosthetic valve ring assembly to annulus tissue of a patient.

### CROSS-REFERENCE TO RELATED APPLICATIONS

The present application is a continuation application of U.S. patent application Ser. No. 18/156,795, filed Jan. 19, 2023, entitled “MITRAL OR TRICUSPID REPAIR SYSTEMS WITH MULTI-DIRECTIONAL ANCHORS,” now U.S. Pat. No. 12,279,955, which is a continuation application of U.S. patent application Ser. No. 15/923,677, filed Mar. 16, 2018, entitled “MITRAL OR TRICUSPID REPAIR SYSTEMS WITH MULTI-DIRECTIONAL ANCHORS,” now U.S. Pat. No. 11,576,779, which claims the benefit of priority under 35 U.S.C. 119 (e) to the filing date of U.S. Provisional Patent Application 62/472,633 filed Mar. 17, 2017, entitled, “IMPROVED MITRAL OR TRICUSPID VALVE REPAIR SYSTEM,” the contents of each of which are incorporated herein by reference in their entirety.

### TECHNICAL FIELD

The present disclosure relates to implantable prosthetic devices. More specifically, the disclosure is directed to an improved prosthetic device implantable by catheter for the treatment of mitral or tricuspid regurgitation.

### BACKGROUND

Mitral Regurgitation is a valvular dysfunction that causes blood volume to flow during systole (during left ventricular contraction) from the left ventricle to the left atrium. In contrast, in a healthy heart, this direction of flow is blocked by the mitral valve. The reverse flow during systole causes pressure to rise in the left atrium, and maintaining a normal cardiac output results in an increased pressure in the left ventricle.

Treating patients with MR (mitral regurgitation) or TR (tricuspid regurgitation) could require valve replacement in order to reduce or eliminate the regurgitation. For many years, the commonly accepted treatment was surgical repair or replacement of the native valve during open heart surgery. In recent years, a trans-vascular technique has been developed for introducing and implanting a prosthetic heart valve using a flexible catheter in a manner that is less invasive than open heart surgery.

In the trans-vascular technique, a prosthetic valve is delivered to the target site (e.g., aortic valve, mitral valve, tricuspid valve, or other valve) through a catheter while the valve is crimped to a low diameter shaft. The valve is then expanded/deployed to a functional size when it is located in the correct position. Examples of such prosthetic valves, and related processes for delivering the valves through a catheter, are described in U.S. Pat. No. 8,518,107, the content of which is hereby incorporated by reference in its entirety.

Advancing the catheter to the target site can be achieved through: (a) The vascular system where a catheter is advanced from the femoral vein/artery, or any other blood vessel that allows access to the target site; (b) Trans-apically where a catheter is advanced through a small incision made in the chest wall and then through the apex; or (c) Trans-atrially where a catheter is advanced through a small incision made in the chest wall and then through the left or right atrium.

### SUMMARY

Embodiments herein are directed to various prosthetic valve ring assemblies for use in repairing cardiac valves suffering from, for example, mitral or tricuspid regurgitation.

A prosthetic valve ring assembly is described herein. In certain embodiments, the prosthetic valve ring assembly can include an outer tube that includes a plurality of windows; and a plurality of anchors positioned inside the outer tube and about a perimeter of the outer tube. The plurality of anchors are configured to be emitted from the plurality of windows to anchor the prosthetic valve ring assembly to annulus tissue of a patient. In some embodiments, the anchors are configured to be emitted in different directions. For example, a first portion of the anchors are configured to be emitted to a ventricular side of the annulus tissue of the patient and a second portion of the anchors are configured to be emitted to an atrial side of the annulus tissue of the patient.

In some embodiments, the anchors are created using a laser cutting technique. In an embodiment, the laser cutting technique includes cutting according to a laser cut pattern to define a plurality of windows through which the plurality of anchors are configured to be emitted.

In some embodiments, the prosthetic valve ring assembly further includes a closure device configured to lock a distal side and a proximal side of the prosthetic valve ring assembly. In some embodiments, the prosthetic valve ring assembly further includes a post adjustment mechanism that includes a flexible connection configured to move an anterior portion of the prosthetic valve ring assembly relative to a posterior portion of the prosthetic valve ring assembly, thereby changing at least one of a size and a geometry of the prosthetic valve ring assembly.

In some embodiments, the prosthetic valve ring assembly further includes a closure device configured to lock a distal side and a proximal side of the prosthetic valve ring assembly; and an unlocking mechanism configured to unlock the closure device, thereby enabling repositioning or retrieval of the prosthetic valve ring assembly from a patient through a catheter. In some embodiments, the prosthetic valve ring assembly further includes one or more bumps positioned on a perimeter of the prosthetic valve ring assembly and configured to apply additional pressure to trigones of the annulus tissue of the patient, thereby providing improved anchoring of the prosthetic valve ring assembly.

### BRIEF DESCRIPTION OF THE DRAWINGS

Aspects, features, benefits and advantages of the embodiments described herein will be apparent with regard to the following description, appended claims, and accompanying drawings where:

FIG. 1 illustrates a top view of an improved mitral valve ring, in accordance with at least one embodiment of the present disclosure.

FIG. 2 illustrates a side view of an improved mitral valve ring, in accordance with at least one embodiment of the present disclosure.

FIG. 3 illustrates a bottom view of an improved mitral valve ring, in accordance with at least one embodiment of the present disclosure.

FIG. 4 illustrates another side view of an improved mitral valve ring, in accordance with at least one embodiment of the present disclosure.

FIG. 5 illustrates a ring laser cut pattern, in accordance with at least one embodiment of the present disclosure.

FIG. 6 illustrates a posterior anchor zone with anchors activated by passive anchor stop features, in accordance with at least one embodiment of the present disclosure.

FIG. 7 illustrates a posterior anchor zone with anchors activated by active anchor stop features, in accordance with at least one embodiment of the present disclosure.

FIG. 8 illustrates the 3D shape of posterior and/or anterior zone anchors, in accordance with at least one embodiment of the present disclosure.

FIG. 9 illustrates the angles of posterior and/or anterior zone anchors, in accordance with at least one embodiment of the present disclosure

FIG. 10 illustrates a perspective view of a closure device, in accordance with at least one embodiment of the present disclosure.

FIG. 11 illustrates a cross-sectional view of a closure device, in accordance with at least one embodiment of the present disclosure.

FIG. 12 illustrates another perspective view of a closure device, in accordance with at least one embodiment of the present disclosure.

FIG. 13 illustrates a top view of a mitral valve ring that includes anchors adjacent to the closure device area, in accordance with at least one embodiment of the present disclosure.

FIG. 14 illustrates an isometric view of a mitral valve ring that includes anchors adjacent to the closure device area, in accordance with at least one embodiment of the present disclosure.

FIG. 15 illustrates a top side view of a mitral valve ring that includes anchors adjacent to the closure device area, in accordance with at least one embodiment of the present disclosure.

FIG. 16 illustrates a top view of an alternative ring assembly including bumps in the trigones region, in accordance with at least one embodiment of the present disclosure.

FIG. 17 illustrates a top view of an alternative ring assembly including bumps in the trigones region with anchors, in accordance with at least one embodiment of the present disclosure.

FIG. 18 illustrates a side view of an alternative ring assembly including bumps in the trigones region with anchors, in accordance with at least one embodiment of the present disclosure.

FIG. 19 illustrates another side view of an alternative ring assembly including bumps in the trigones region with anchors, in accordance with at least one embodiment of the present disclosure.

FIG. 20 illustrates yet another side view of an alternative ring assembly including bumps in the trigones region with anchors, in accordance with at least one embodiment of the present disclosure.

FIG. 21 illustrates an alternative mitral valve ring with one or more post adjustment mechanisms, in accordance with at least one embodiment of the present disclosure.

FIG. 22 illustrates another alternative mitral valve ring with one or more post adjustment mechanisms, in accordance with at least one embodiment of the present disclosure.

FIG. 23 illustrates a laser cut layout for a tricuspid ring, in accordance with at least one embodiment of the present disclosure.

FIG. 24 illustrates a 3D shape for a tricuspid ring, in accordance with at least one embodiment of the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

When implanting a replacement valve (e.g., an aortic valve, mitral valve, tricuspid valve, or other valve), the replacement valve can include a prosthesis attachment. The prosthesis can be configured to secure the replacement valve in a patient's heart. An example of such a prosthesis is the AMEND™ Mitral Valve Repair Annuloplasty ring developed by Valcare Medical. The AMEND™ ring is a D-shaped ring configured to emulate the total valve replacement for patients who suffer from Mitral Regurgitation (MR, as described above). Additional detail related to prosthetic valves for mitral or tricuspid valve replacement can be found in U.S. patent application Ser. No. 14/891,189 entitled “Transcatheter Prosthetic Valve for Mitral or Tricuspid Valve Replacement,” filed May 22, 2013, the content of which is incorporated herein by reference in its entirety.

As described herein, a prosthetic ring can be configured to include various anchor zones on a posterior side. The anchor zones are positioned to emit anchors in different directions and/or angles from two or more windows on the cross section of the ring. For example, a portion of the anchors may be emitted to the ventricular side of the annulus tissue, and a portion of the anchors may be emitted to the atrial side of the annulus tissue. Such an arrangement can provide for improved anchoring of the ring into the annulus tissue as compared to conventional mitral valve rings.

It should be noted that, while the following detailed description of the figures is directed to a prosthetic ring configured to anchor a replacement valve such as the AMEND™ ring as described above, the techniques, ideas, and processes described herein can be applied to any mitral valve ring.

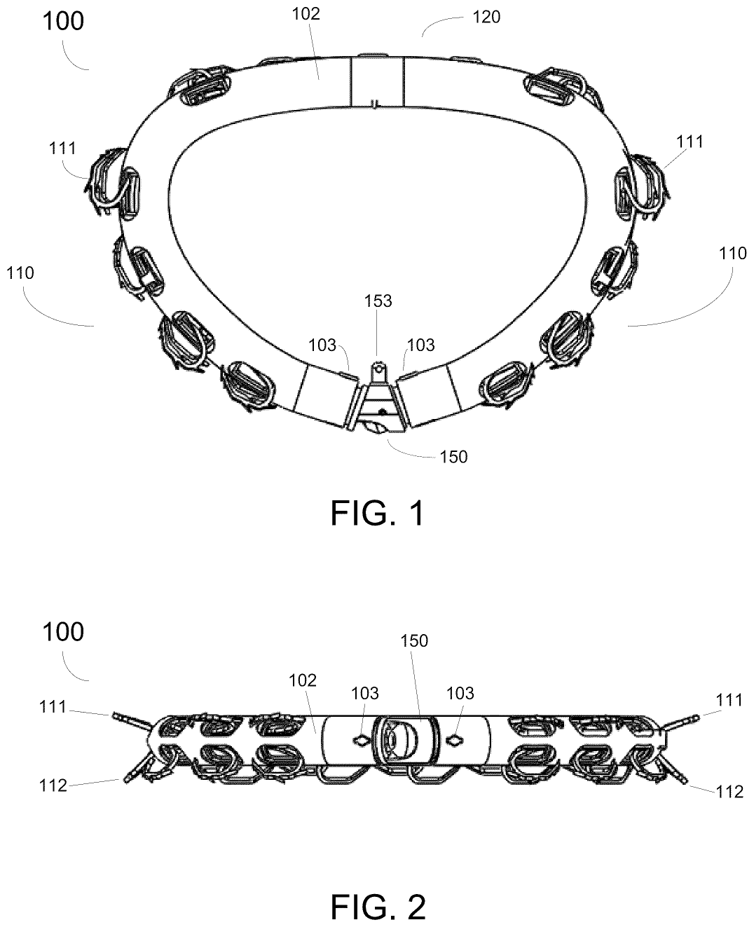

FIGS. 1-4 illustrate various views of an improved mitral valve ring 100. In certain implementations, the ring 100 can include posterior side anchor zones that are configured to emit anchors in different directions and/or angles from two or more windows on the cross section of the ring. In some examples, a portion of the anchors are configured to be emitted to the ventricular side of a patient's annulus tissue, while a second portion of the anchors are configured to be emitted to the atrial side of the annulus tissue. Thus, two or more anchors can be emitted from one window in different directions.

FIG. 1 illustrates a top view of the ring assembly 100. The ring assembly 100 can include an outer tube 102 that defines the shape of the ring assembly. The outer tube 102 can include one or more windows about its circumference as well as one or more windows in its cross-section. One or more anchors can be positioned and configured to extend through these windows. The ring assembly 100 can also include a set of posterior zone anchors 110. As shown in FIG. 1, the posterior zone anchors 110 are generally positioned on the posterior of the ring assembly 100. The ring assembly 100 can further include anterior zone anchors 120 generally positioned on the anterior of the ring assembly.

Each anchor zone (including both posterior zone anchors 110 and anterior zone anchors 120) can include atrial side anchors 111 as well as ventricular side anchors 112 (not shown in FIG. 1 but illustrated in, for example, FIG. 2). As noted above, in certain implementations, upon insertion of the ring assembly 100, the atrial side anchors 111 can be configured to be emitted into the atrial side of the patient's annulus tissue and the ventricular side anchors can be configured to be emitted into the ventricular side of the patient's annulus tissue.

As further shown in FIG. 1, the ring assembly 100 can include a closure device 150. The closure device 150 can be configured to lock a distal end of the ring assembly 100 to a proximal end of the ring assembly. The closure device 150 can also be designed and configured to removably attach to a delivery system for the valve replacement implant. For example, in certain implementations, the closure device 150 can include a pivot pin 153 that is configured to removably attach the ring assembly 100 to a delivery device as well as provide for rotation of the ring assembly when emitted from the delivery device. For example, the pivot pin 153 can be configured to provide for 90 degrees of rotation of the ring assembly 100. In other implementations, the pivot pin can be configured to provide for additional ranges of rotation such as 75-105 degrees of rotation, 60-120 degrees of rotation, and other similar ranges of rotation.

The ring assembly 100 can further include a set of pins 103. The pins 103 can be positioned and configured to connect the outer tube 102 to the closure device 150. In certain implementations, the pins 103 can be further positioned and configured to provide for routing of sutures as well as to function as a pulley while providing for rotation of a suture (e.g., 90 degree rotation) with minimal friction.

FIG. 2 illustrates a first side view of the ring assembly 100, shown from the posterior and further illustrating the closure device 150. FIG. 2 further illustrates the alternate emitting directions of the atrial side anchors 111 in an outward and upward direction relative to a plane of the ring assembly 100 and the ventricular side anchors 112 in an outward and downward direction relative to the plane of the ring assembly 100.

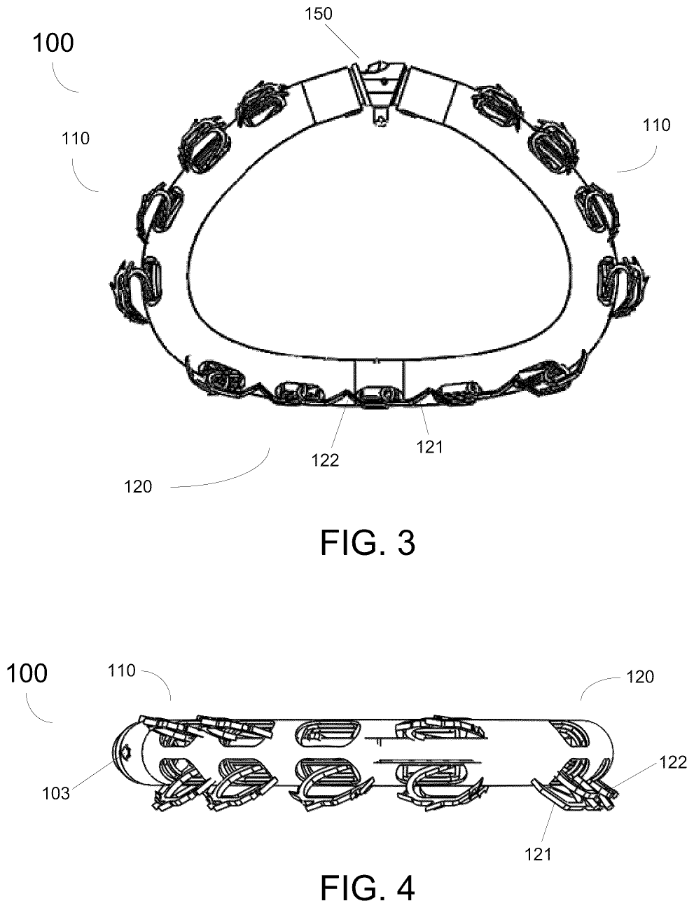

FIG. 3 illustrates a bottom view of the ring assembly 100. In contrast to FIG. 1, FIG. 3 illustrates the ventricular side anchors 112. FIG. 3 further illustrates additional anterior side anchors 120 that were obscured from view in FIG. 1.

Additionally, in certain embodiments, various anchors can be configured such that portions of the anchors can cross one another in different directions, thereby creating a closed loop and stapling effect of the tissue. For example, anchors 121 and 122 as shown in FIG. 3 are configured to cross one another in different directions, thereby having a stapling effect on the adjacent tissue, securely locking the ring assembly 100 into position.

FIG. 4 illustrates a second side view of the ring assembly 100, shown further illustrating the crossing anchors 121 and 122.

The raw materials of the ring assembly 100 and various components included therein can be selected from various materials, such as various polymers, shape memory materials such as Nitinol, metals such as stainless steel, or other similar materials safe for implanting into or adjacent to living tissue. In certain implementations, the ring assembly 100 can include a combination of two or more different materials, such as stainless steel 316/316L and Nitinol. This combination is provided by way of example only, and other materials can be used alternately or additionally.

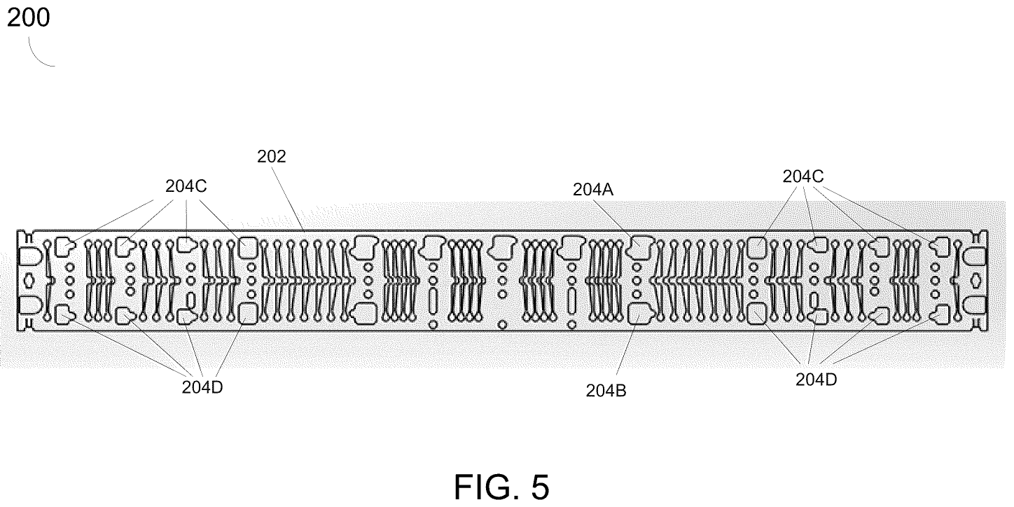

In order to create the ring assembly 100 as described above, the outer tube 102 can be manufactured such that multiple windows are defined. The windows can be positioned to facilitate deployment of the various anchors as described above. FIG. 5 illustrates an example cut pattern 200 for creating the outer tube 102 as described above. For example, the pattern 200 shows an example cut pattern of a ring tube 202 with several windows 204A, 204B, 204C and 204D that allow deployment of anchors in different directions. For example, anchors can be emitted in the atrial side from windows 204D, and anchors can be emitted from the ventricular side from windows 204C.

In an alternate embodiment, more than one anchor can be emitted from one window with one or more anchors in the atrial side and one or more anchors in ventricular side. In another embodiment, one or more anchors can be emitted from different windows in the same direction. For example, two windows (e.g., 204A and 204B) can be configured to provide for simultaneous deployment of anchors towards the ventricular side.

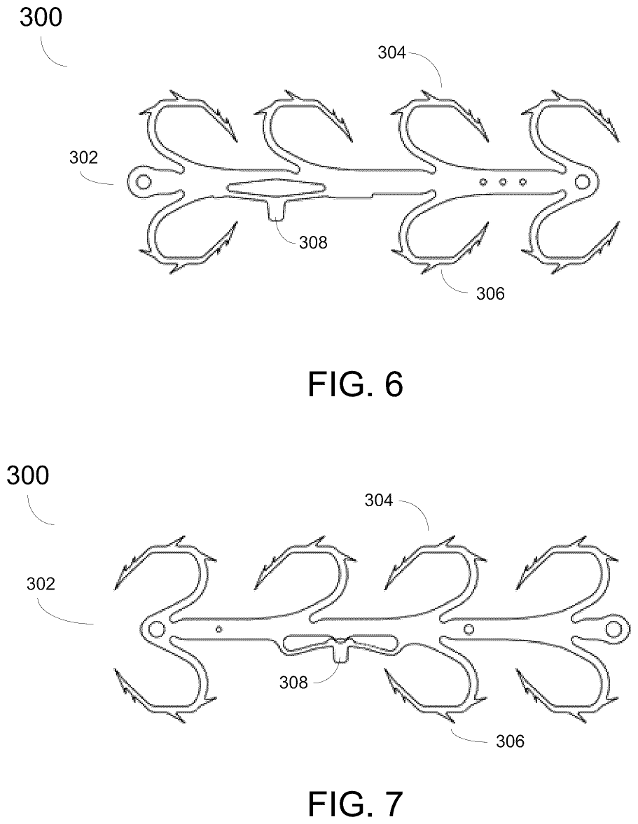

FIGS. 6 and 7 illustrate example cut patterns (e.g., laser cut patterns) for the anterior and/or posterior zone anchors such as posterior zone anchors 110 and anterior zone anchors 120 as described above. Additionally, as noted above, anchors can be emitted in both the atrial and ventricle directions, such as atrial anchors 111 and ventricle anchors 112 as described above.

To achieve such a configuration, cut pattern 300 as shown in FIGS. 6 and 7 can be used to create one or more anchor zones 302. The cut pattern can define a set of atrial anchors 304 and a set of ventricle anchors 306 within the anchor zone 302. The cut pattern 300 can also define an anchor stop feature 308. The anchor stop feature 308 can be configured to lock the anchor zone 302 into, for example, the outer tube of a ring assembly to prevent unintentional movement of the anchor zone.

In certain implementations, the anchor stop feature 308 can be a passive feature that is activated as a result of a force exerted on the anchor zone 302 (e.g., a pulling force) causing a bending or other change to the geometry of the anchor stop feature 308. In other embodiments, the anchor stop feature 308 can include an associated activation mechanism that facilitates activation of the anchor stop feature. For example, the activation mechanism can be an activation pulley that prevents the anchor stop feature 308 from bending of otherwise deforming such that a portion of the anchor stop feature exits through a corresponding hole on the outer tube of the ring assembly. Such an arrangement can lock the anchor zone 302 into position as the anchor stop feature 308 prevents any relative movement. In some examples, pulling on the activator mechanism and removing it from the designated location allows the anchor stop feature 308 to bend and allows relative movement of the anchor zone 302 in relation to the outer tube of the ring assembly.

In certain implementations, the anchor stop feature 308 can be located in either the ventricular side or atrial side of the anchor zone 320 and/or outer tube. Additionally, in various embodiments, the anchor stop feature 308 can be positioned in any location along the anchor zone 302. The position as shown in FIGS. 6 and 7 is shown by way of example only.

It should be noted that laser cutting the ring assemblies as described herein is provided by way of example only, and additional manufacturing techniques can be used. For example, a stamping process can be used to stamp the cut patterns as described above.

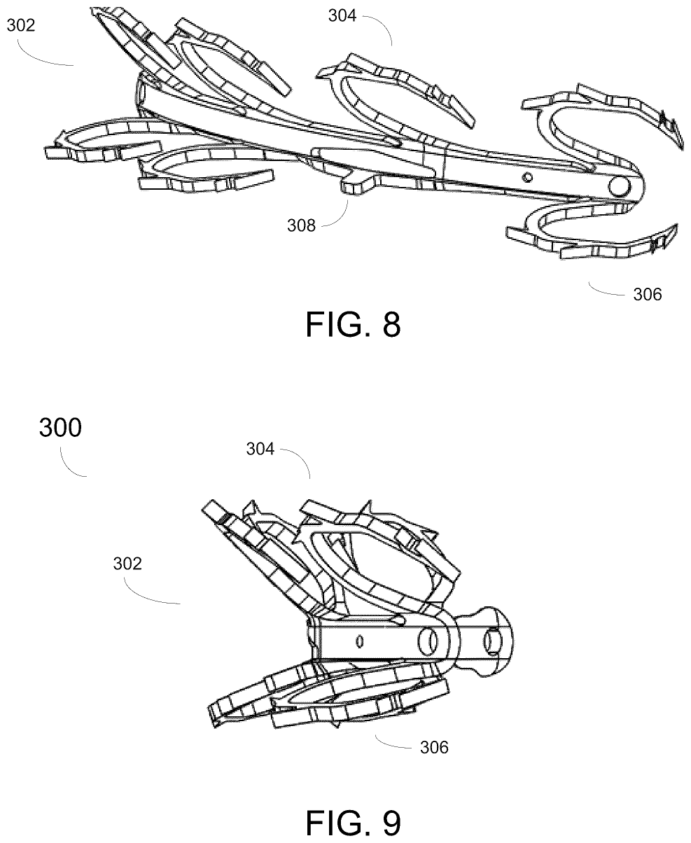

FIGS. 8 and 9 illustrate the cut patterns 300 as described above in regard to FIGS. 6 and 7 that have been, for example, heat treated and to include various bends and curves, thereby defining a specific three-dimensional shape for the anchor zones 302. For example, the anchor zones 302 can be heat-treated and bent at an angle selected from a range of angles. For example, in certain implementations the range of angles can be from zero degrees (e.g., no additional bending) to 135 degrees. As shown in FIGS. 8 and 9, the ventricle anchors 306 can be heat-treated to 45 degrees, and the atrial anchors 304 can be heat-treated to 60 degrees.

In addition, the anchor zone can have a 3D shape that fits the zone location in the final ring assembly. For example, the posterior anchor zones can be curved to fit the posterior curvature of the ring assembly, and the anterior anchor zones can be curved to fit the anterior curvature of the ring assembly.

It should be noted that heat-treated bending and curving is provided by way of example only. Depending upon the type of material being used and the design of the individual components such as the anchor zones, alternative bending and curving techniques can be used to form the anchor zones to the geometry of the final ring assembly.

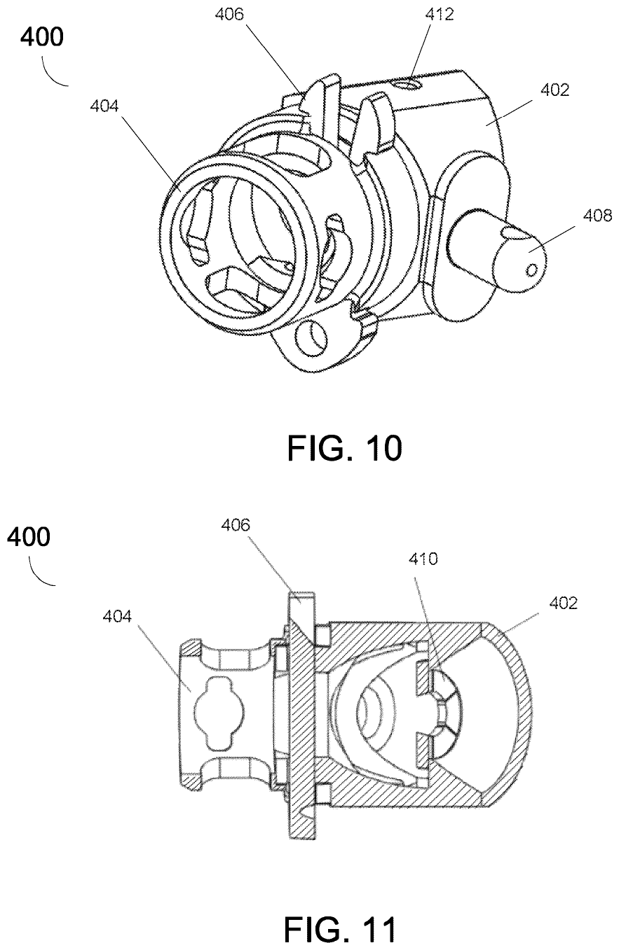

FIGS. 10-12 illustrate detailed views of a closing mechanism 400 for a ring assembly, such as closing mechanism 150 as described above in regard to ring assembly 100. In certain implementations, a closing mechanism can include a female component configured to lock to a male component. For example, FIGS. 10-12 illustrate various views and embodiments of the female component 402. As shown in FIG. 10, the female component 402 can include a locking mechanism for releasably attaching to a male component or cup 404.

The female component 402 can further include an unsnapping pin 406. The unsnapping pin can be positioned and configured to unsnap or otherwise disconnect the female component 402 from the cup 404 of the ring assembly. Additionally, the female component 402 can include a pivot pin 408 for attaching the female component to an outer tube of the ring assembly. Additionally, the pivot pin 408 can be configured to function as an interface between the ring assembly and a delivery system, similar to pins 103 as described above.

FIG. 11 illustrates a cross-section of the female component 402. As shown in the cross-sectional view, the female component 402 can include a disk 410. The disk 410 can be positioned to abut an end of the cup 404 when inserted into the female component 402 to provide for locking of the male component to the female component. In some examples, the disk 410 can be held in position with a cover.

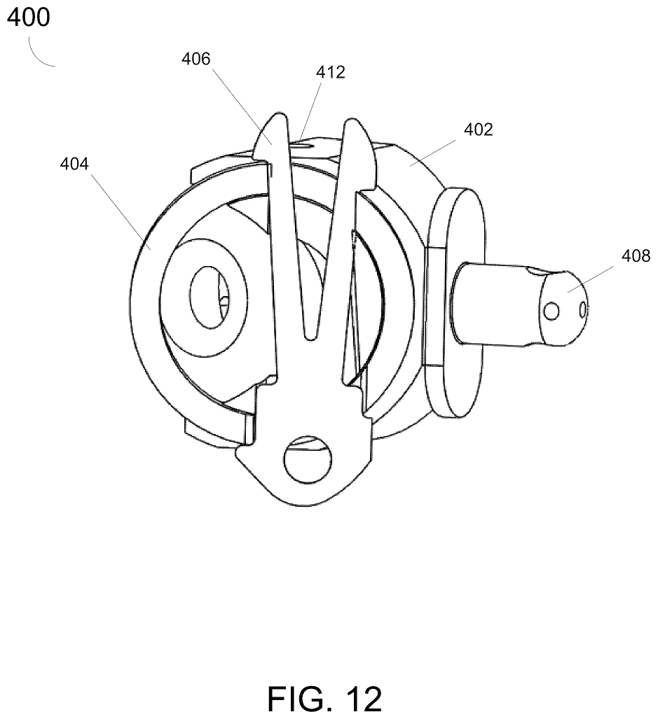

FIG. 12 illustrates a view of the locking mechanism 400 with a portion of the cup 404 removed, showing additional detail of the unsnapping pin 406. As shown in FIG. 12, the unsnapping pin 406 can be designed with two individual fingers extending from a central point such that, upon exerting a force (e.g., a squeezing force) upon an end of the fingers opposite the central point, the fingers deflect about the point. Upon deflection of the unsnapping pin 406, the unsnapping pin can be removed, thereby releasing the male component (e.g., cup 404) from the female component 402.

In certain implementations, the disk 410 and the unsnapping pin 406 can be made from a shape memory alloy such as Nitinol. In some example, the other components of the female component can be made from various metallic materials such as stainless steel, aluminum, Nitinol, and titanium.

As noted above, the cup 404 and the female component 402 are attached together with the unsnapping pin 406. If properly inserted and positioned, the unsnapping pin 406 can visually verify that the two parts are well-attached and cannot open unintentionally. Conversely, upon activation of the unsnapping pin 406 by pulling it in a specific direction, it allows separation of the two components. By doing this, the ring close structure is compromised and the closed shape becomes open and allows retrieval of the implant into the delivery system.

In some embodiments, the female component 402 can have one or more gold markers 412 that provide for confirmation of locking of the cup 404 and the female component during a clinical procedure.

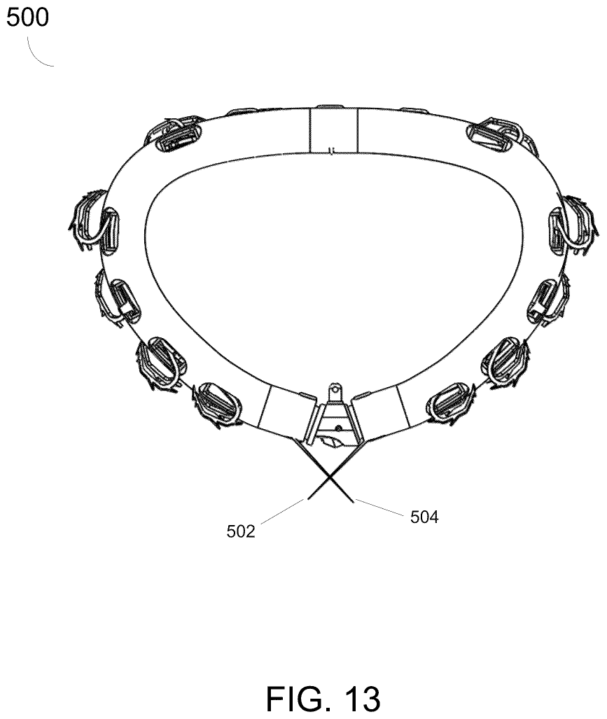

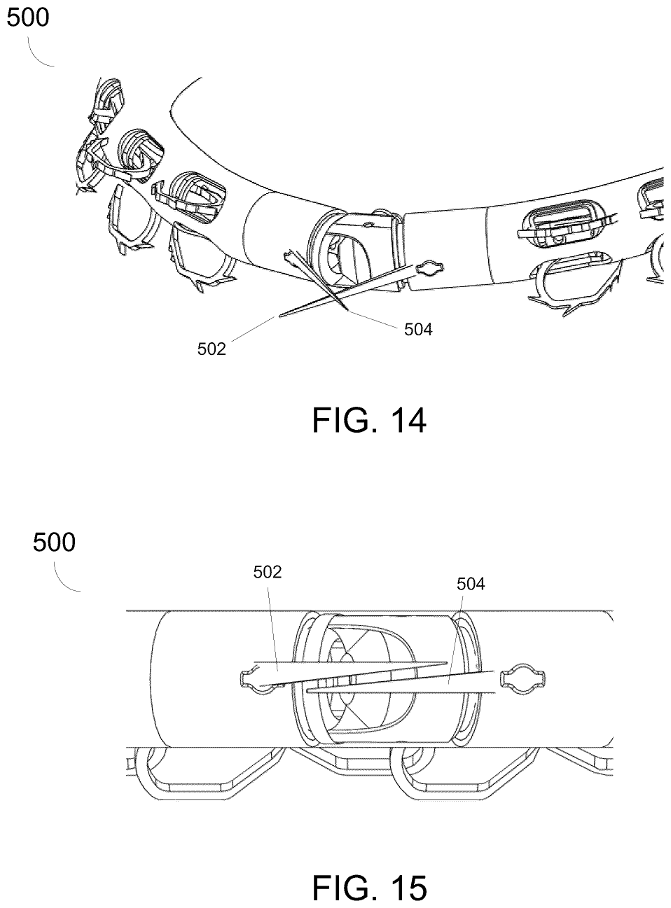

FIGS. 13-15 illustrate various views of a ring assembly 500. Similar to ring assembly 100 as described above, the ring assembly 500 can include an outer tube, a set of posterior zone anchors, a set of anterior zone anchors, and a closure device 150. However, the ring assembly 500 as shown in FIGS. 13-15 can further include additional anchors 502 and 504 positioned adjacent to the closure device and configured to improve attachment of the ring assembly 500 to the patient's posterior annulus tissue. In certain implementations, the anchors 502 and 504 can be configured to be emitted in different directions and/or angles from one or more windows on the cross section of the outer tube in the area of the closure device. In some examples, one or more additional anchors can be configured to be emitted to the ventricular side of the annulus tissue, and one or more of the additional anchors can be configured to be emitted to the atrial side of the annulus tissue. In some embodiments, two or more additional anchors can be configured to be emitted from one window in the outer tube in different directions to each other. In some embodiments, the anchors can include additional features such as hooks, barbs, or other similar features for increasing durability and preventing detachment.

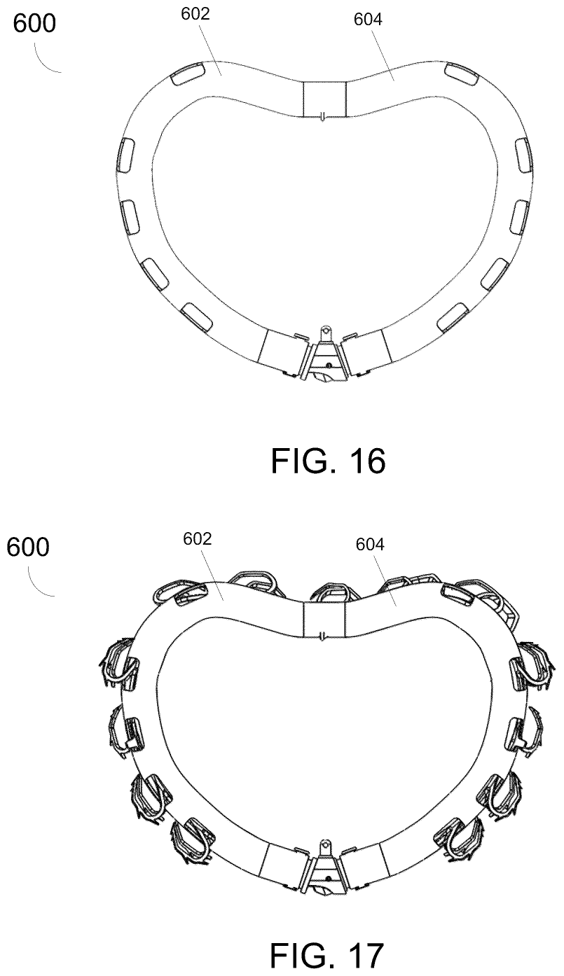

In certain implementations, a ring assembly can further include bumps or other similar protrusions in the trigones area of the ring assembly. For example, as shown in FIGS. 16 and 17, a ring assembly 600 (shown without anchors in FIG. 16 and with anchors in FIG. 17) can include bumps 602 and 604 positioned on the portion of the ring assembly that will be adjacent to the fibrous trigones region of a patient's heart. The bumps 602 and 604, or other similar protrusions or added features, can provide for added contact between the ring assembly 600 and the patient's trigones region of the annulus tissue. In some implementations, achieving such contact can provide for better contact between the ring assembly 600 and the annulus tissue as compared to a ring assembly without the bumps in the trigones region. Similar to the ring assemblies as described above, various patterns and directions of anchors can be used to improve anchoring the ring assembly 600 to the annulus tissue as well.

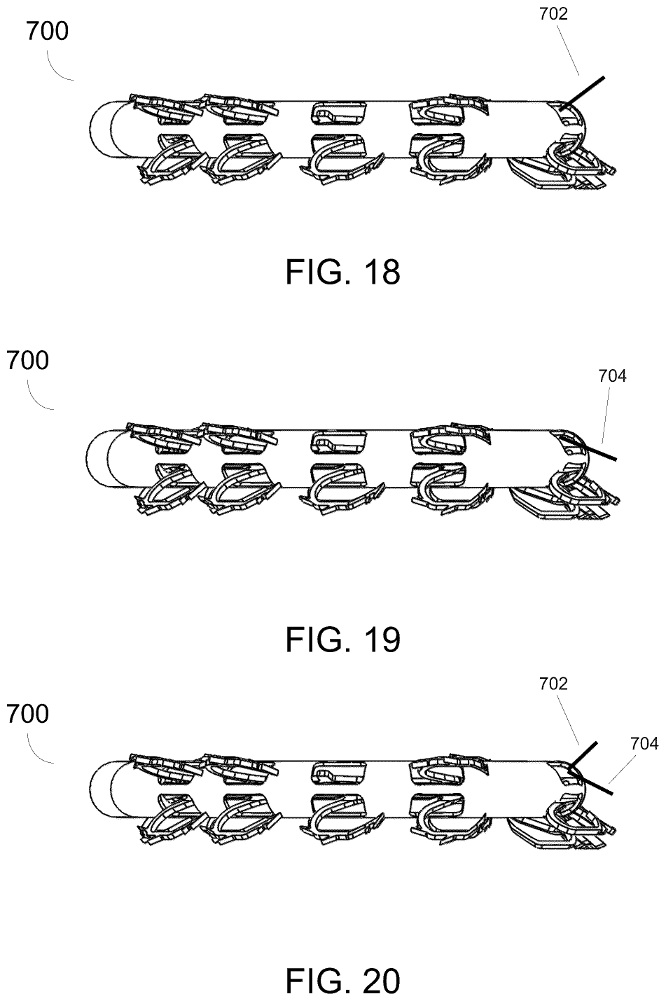

In certain implementations, additional anchors can be beneficial for providing additional anchoring points for a ring assembly. In some examples, a single anchor or set of additional anchors can be emitted from an existing window in an outer tube of a ring assembly providing a more robust anchoring point as compared to the curved anchors as described above. For example, as shown in FIGS. 18-20, ring assembly 700 can include additional anchors configured to provide a more robust anchoring point. As shown in FIG. 18, an additional anchor 702 can be emitted from a window for providing an additional anchoring point on the atrial side of the ring assembly 700. FIG. 19 illustrates an additional anchor 704 being emitted from a similar window for providing an additional anchoring point on the ventricular side of the ring assembly 700. FIG. 20 illustrates a set of two or more additional anchors emitting from a single window. In this example, additional anchors 702 and 704 provide additional anchoring into the atrial side and the ventricular side of the ring assembly 700 respectively.

In some implementations, resizing the ring assembly can be desirable as a single ring assembly can accommodate an additional range of patients and valve sizes. For example, a ring assembly can include a flexible connection that is laser cut from a similar material as the outer tube of the ring assembly. The flexible connection can be configured to contract and/or expand to allow for changing the size of the ring before, during, and/or after implantation of the ring assembly. In some examples, the ring assembly can be manufactured from separate segments or components that are configured to be attached together with a mechanism or device that controls expansion and/or contraction of each side of a ring assembly separately or simultaneously.

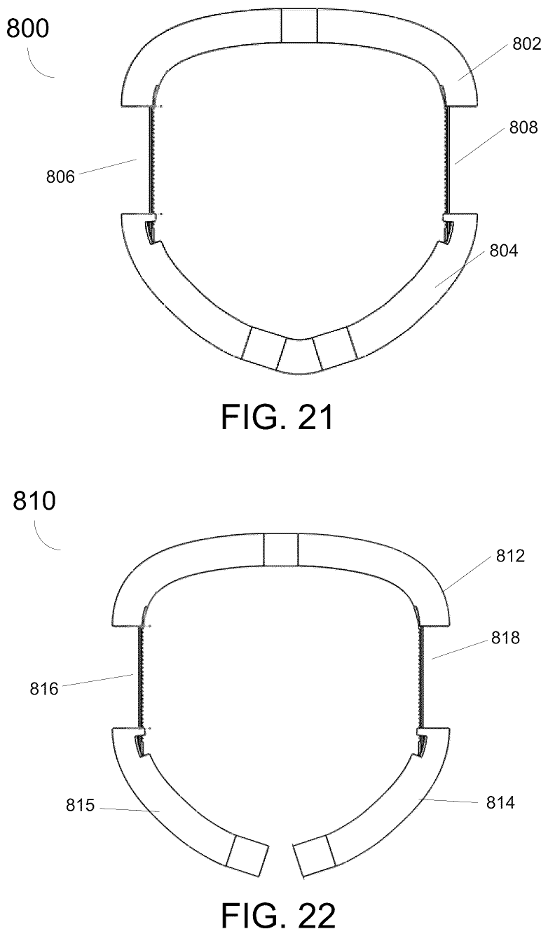

FIG. 21 illustrates a ring assembly 800 that is configured to be resizable as described above. The ring assembly 800 can include an anterior portion 802 and a posterior portion 804. The anterior portion 802 and the posterior portion 804 can be connected together with a set of adjustable components 806 and 808. The adjustable components 806 and 808 can be made from a flexible material configured to expand and/or contract, thereby changing the overall size of the ring assembly 800. In certain implementations, the anterior portion 802 and/or the posterior portion 804 can include an adjustment mechanism that is configured to interact with the adjustable components 806 and 808 to change the size, shape and/or geometry of the ring assembly 800. For example, the adjustment mechanisms can include a ratcheting feature that is configured to interact with the adjustable components 806 and 808 to change the size of the ring assembly 800.

It should be noted that a ratcheting feature is provided by way of example only. Additional adjustment mechanisms such as friction-based holding devices, snap-based devices, winding devices, and other similar adjustment devices can be used. Additionally, it should be noted that two adjustable components 806 and 808 are shown by way of example. In additional implementations, various numbers of adjustable components can be used. For example, a single adjustable component can be included on one side of the ring assembly.

FIG. 22 illustrates a ring assembly 810 that is configured to be resizable, similar to ring assembly 800 as described above. However, the ring assembly 810 can include an anterior portion 812 and two posterior portions 814 and 815. Such an arrangement can provide for additional flexibility and sizing options when implanting the ring assembly 810.

In some implementations, the anterior portion 812 can be connected to the posterior portion 815 with a first adjustable component 816. Similarly, the anterior portion can be connected to the posterior portion 814 with a second adjustable component 818. The posterior portions 814 and 815 can be configured to releasably attach to one another via a closure device such as closure device 150 as described above.

The ring assemblies as described above can be designed and shaped for various functions such as mitral valve replacement. However, a similar ring assembly can be designed and constructed for tricuspid valve replacement as well. However, a tricuspid ring can be designed with additional features such as a release zone positioned on the ring assembly at a location that will be adjacent to a patient's atrioventricular node or valves.

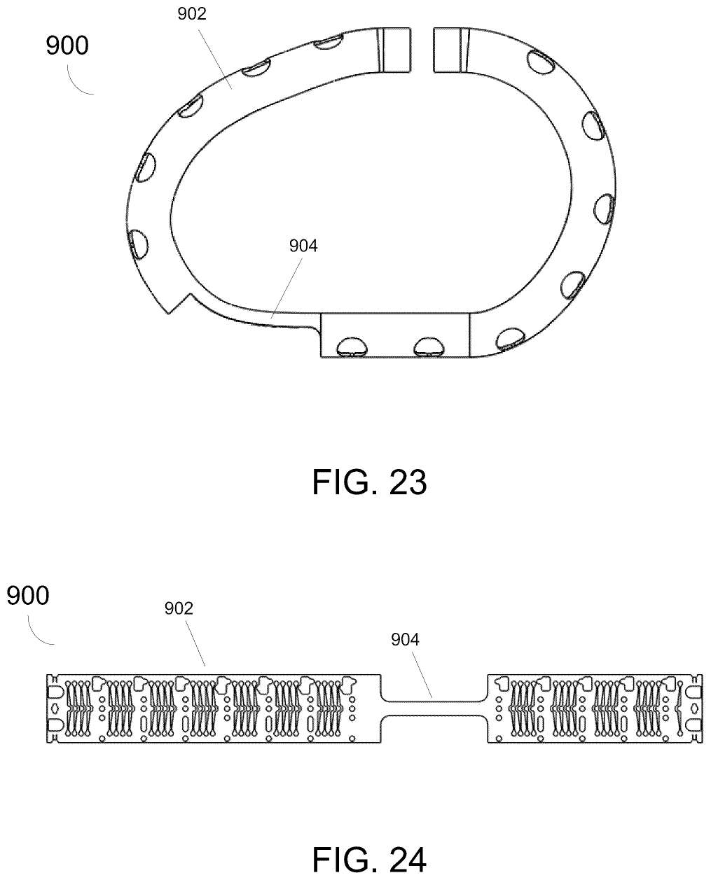

FIGS. 23 and 24 illustrate an example tricuspid ring assembly 900 (FIG. 23 illustrating a 3D view of the assembly, while FIG. 24 illustrates a flat cut pattern view). The outer tube 902 of the ring assembly 900 can include multiple windows through which anchors can be emitted as described above. However, the ring assembly 900 can further include a release zone 904 configured and positioned at a location that will be adjacent to a patient's atrioventricular node or valves when the ring assembly is implanted.

In certain implementations, the release zone 904 does not have any anchors. Rather, the alternate shape and profile of the release zone provides for interference between the ring assembly 900 and the patient's atrioventricular node or valves, thereby securing the ring assembly in position.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A prosthetic valve ring assembly for repairing a defective heart valve, the prosthetic valve ring assembly comprising:
an outer tube comprising a plurality of windows, the outer tube configured to transition between a delivery state for catheter-based delivery and a deployed state for implantation at the defective heart valve; and
a plurality of anchors positioned inside the outer tube and around a perimeter of the outer tube in the delivery state, the plurality of anchors configured to be emitted through the plurality of windows to anchor the prosthetic valve ring assembly to annulus tissue of the defective heart valve such that a first portion of the plurality of anchors is configured to be emitted in a first direction to a ventricular side of the annulus tissue and a second portion of the plurality of anchors is configured to be emitted in a second direction to an atrial side of the annulus tissue, the first direction different than the second direction.

2. The prosthetic valve ring assembly of claim 1, further comprising a closure device configured to lock a distal end and a proximal end of the prosthetic valve ring assembly in the deployed state.

3. The prosthetic valve ring assembly of claim 1, further comprising an unsnapping pin configured to unlock the closure device to separate the distal end and the proximal end of the prosthetic valve ring assembly, thereby providing for repositioning or retrieval of the prosthetic valve ring assembly from the patient through a catheter.

4. The prosthetic valve ring assembly of claim 1, wherein the plurality of windows comprises a first plurality of windows oriented toward the ventricular side of the annulus tissue and a second plurality of windows oriented toward the atrial side of the annulus tissue.

5. The prosthetic valve ring assembly of claim 4, wherein the first portion of the plurality of anchors is configured to be emitted through the first plurality of windows and the second portion of the plurality of anchors is configured to be emitted through the second plurality of windows.

6. The prosthetic valve ring assembly of claim 4, wherein the first plurality of windows are positioned on the outer tube directly below corresponding windows of the second plurality of windows.

7. The prosthetic valve ring assembly of claim 1, wherein the plurality of anchors comprises an anterior zone anchor configured to engage an anterior portion of the annulus tissue and a posterior zone anchor configured to engage a posterior portion of the annulus tissue.

8. The prosthetic valve ring assembly of claim 7, wherein the anterior anchor zone comprises the first portion and the second portion of the plurality of anchors and the posterior anchor zone comprises a third portion configured to be emitted in the first direction to the ventricular side of the annulus tissue and a fourth portion configured to be emitted in the second direction to the atrial side of the annulus tissue.

9. The prosthetic valve ring assembly of claim 1, further comprising a second anchor oriented in a third direction, the third direction different than the first direction and the second direction.

10. The prosthetic valve ring assembly of claim 9, further comprising a third anchor oriented in a fourth direction, the fourth direction different than the first direction and second direction and opposite the third direction.

11. A method for implanting a prosthetic valve ring assembly for repairing a defective heart valve, the method comprising:
delivering the prosthetic valve ring assembly to the defective heart valve in a delivery state, the prosthetic valve ring assembly comprising an outer tube comprising a plurality of windows and a plurality of anchors positioned inside the outer tube and around a perimeter of the outer tube;
causing the outer tube to transition from the delivery state to a deployed state; and
causing deployment of the plurality of anchors through the plurality of windows to anchor the prosthetic valve ring assembly to annulus tissue of the defective heart valve such that a first portion of the plurality of anchors is emitted in a first direction to a ventricular side of the annulus tissue and a second portion of the plurality of anchors is emitted in a second direction to an atrial side of the annulus tissue, the first direction different than the second direction.

12. The method of claim 11, wherein the prosthetic valve ring assembly further comprises a closure device and the method further comprises causing the closure device to lock a distal end and a proximal end of the prosthetic valve ring assembly in the deployed state.

13. The method of claim 11, wherein the prosthetic valve ring assembly is delivered via a catheter delivery system.

14. The method of claim 11, wherein the plurality of windows comprises a first plurality of windows oriented toward the ventricular side of the annulus tissue and a second plurality of windows oriented toward the atrial side of the annulus tissue,

15. The method of claim 14, wherein the first plurality of windows are positioned on the outer tube directly below corresponding windows of the second plurality of windows.

16. The method of claim 14, wherein causing deployment of the plurality of anchors comprises emitting the first portion of the plurality of anchors through the first plurality of windows and emitting the second portion of the plurality of anchors through the second plurality of windows.

17. The method of claim 11, wherein the plurality of anchors comprises an anterior zone anchor configured to engage an anterior portion of the annulus tissue and a posterior zone anchor configured to engage a posterior portion of the annulus tissue.

18. The method of claim 17, wherein the anterior anchor zone comprises the first portion and the second portion of the plurality of anchors and the posterior anchor zone comprises a third portion configured to be emitted in the first direction to the ventricular side of the annulus tissue and a fourth portion configured to be emitted in the second direction to the atrial side of the annulus tissue.

19. The method of claim 11, further comprising a second anchor oriented in a third direction, the third direction different than the first direction and the second direction.

20. The method of claim 19, further comprising a third anchor oriented in a fourth direction, the fourth direction different than the first direction, second direction, and third direction.
